(19) United States
(12) Patent Application Publication
(10) Pub. No.: US 2026/0106026 A1
(43) Pub. Date: Apr. 16, 2026

(54) CONTINUOUS GLUCOSE DYNAMICS INDICES AS DIAGNOSTIC MARKERS FOR PROGRESSION TO STAGES AND TYPES OF DIABETES

(71) Applicant: UNIVERSITY OF VIRGINIA PATENT FOUNDATION, Charlottesville, VA (US)

(72) Inventors: Boris Kovatchev, Charlottesville, VA (US); Eslam Montaser, Charlottesville, VA (US)

(21) Appl. No.: 19/224,171

(22) Filed: May 30, 2025

Related U.S. Application Data
(60) Provisional application No. 63/707,729, filed on Oct. 15, 2024.

(51) Int. Cl. G16H 50/20; G06F 17/18

(52) U.S. Cl. CPC G16H 50/20; G06F 17/18

(57) ABSTRACT

According to one embodiment, a method, computer system, and computer program product for diagnosing and tracking progression of and categories of a condition in a subject. The embodiment may include identifying a concentration level of a metabolite corresponding to a subject. The embodiment may also include transforming the concentration level into a symbol from a set of symbols, wherein the symbol represents a category of concentration levels. The embodiment may further include identifying a matrix of transition probabilities between symbols in a series of symbols. The embodiment may also include identifying an entropy rate of concentration level state categories based on the matrix. The embodiment may further include determining a diagnostic state for the subject based on the entropy rate.

### CROSS REFERENCE TO RELATED APPLICATIONS

This application claims priority to and incorporates by reference herein U.S. Provisional Patent Application Ser. No. 63/707,729 filed on Oct. 15, 2024, and entitled Continuous Glucose Dynamics Indices as Diagnostic Markers for Progression to Stages and Types of Diabetes.

### BACKGROUND

The present invention relates generally to the field of computing, and more particularly to medical analytics and diagnostics.

Medical professionals have long understood the importance of comprehensively testing and understanding their patients' condition. However, the advent of new hardware has enabled continuous, often portable monitoring of various metabolic data. Such data has enabled new approaches to medical analytics and diagnostics, allowing for great advancements in research, complex diagnostics, and advanced treatment methods like automated insulin delivery.

### SUMMARY

According to one embodiment, a method, computer system, and computer program product for diagnosing and tracking progression of and categories of a condition in a subject. The embodiment may include identifying a concentration level of a metabolite corresponding to a subject. The embodiment may also include transforming the concentration level into a symbol from a set of symbols, wherein the symbol represents a category of concentration levels. The embodiment may further include identifying a matrix of transition probabilities between symbols in a series of symbols. The embodiment may also include identifying an entropy rate of concentration level state categories based on the matrix. The embodiment may further include determining a diagnostic state for the subject based on the entropy rate.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 depicts an exemplary computing device or computer system with several components.

FIG. 2 is an exemplary flowchart of a method for monitoring levels of a metabolite for use as diagnostic markers.

FIG. 3 is an exemplary flowchart for an alternate method of monitoring levels of a metabolite for use as diagnostic markers through use of Poincaré plot geometry.

FIG. 4 is an exemplary table of potential alphabets representing different categories of data for continuous glucose monitoring (CGM).

FIG. 5 depicts an exemplary process for dynamic CGM and indexing of states for diagnostic purposes.

FIG. 6 is a table reflecting clinical and demographic characteristics of seven different data sets used for analysis. Abbreviations: CLC, closed-loop control; HbA1c, glycated hemoglobin; MDI, multiple daily injections; NR, not reported; SAP, sensor-augmented pump.

FIG. 7 is an explanatory example of encoding a daily CGM profile. The symbolic representation for this CGM daily profile can be represented as a string series: “cccccccccddddeeefffffffffffffdccdeeeffeeeeeeeeeddefggggggffffeeddccccccccddeefgggggffgggff eddddd” with 8 symbols, and a 96-word size.

FIGS. 8A-C portray exemplary ROC curves for three different cases of discrimination to evaluate the discriminative power of the ER and the Area of Poincaré plot of the CGM process, S, as biomarkers. FIG. 8A presents ROC curves for the discrimination between healthy and individuals with diabetes. FIG. 8B presents ROC curves for the discrimination between type 1 diabetes and type 2 diabetes individuals. FIG. 8C presents ROC curves for the discrimination between individuals with low risk and high risk of developing type 1 diabetes.

FIG. 9 presents exemplary scatterplots with a standard error for the entropy rate criterion and the area of a fitting ellipse (mg2/dL2) of a Poincaré plot for the different datasets.

FIG. 10A presents an illustrative example of a transition probability matrix for four different individuals.

FIG. 10B presents an illustrative example of a transition probability matrix for four different individuals.

FIG. 10C presents an illustrative example of a transition probability matrix for four different individuals.

FIG. 10D presents an illustrative example of a transition probability matrix for four different individuals.

FIG. 11A presents a Poincaré plot for a respective one of two individuals.

FIG. 11B presents a Poincaré plot for a respective one of two individuals.

FIG. 12 presents an illustrative example of a Poincaré plot, a fitting ellipse of the Poincaré plot, and line segments useful for calculating the area of the fitting ellipse.

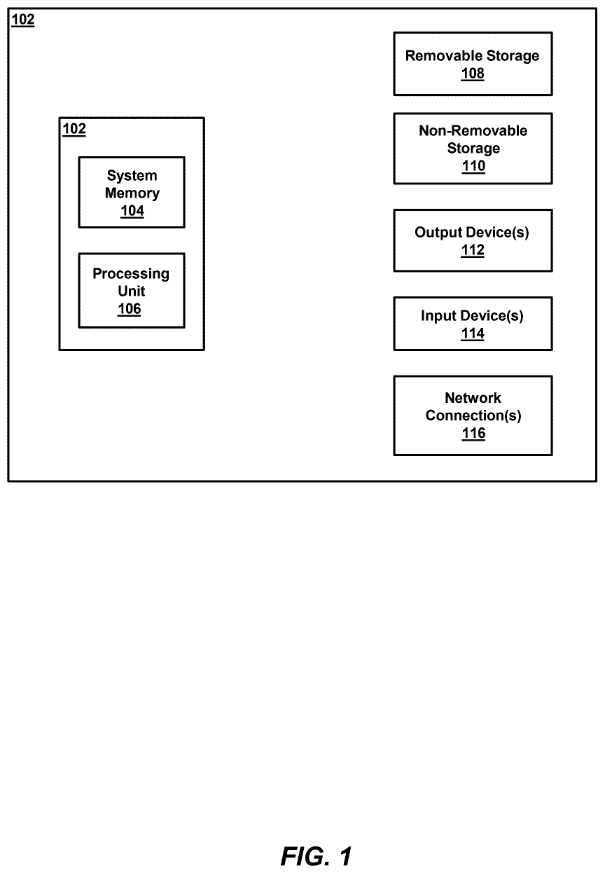

It should be appreciated that the logical operations described herein with respect to the various figures may be implemented (1) as a sequence of computer implemented acts or program modules (i.e., software) running on a computing device (e.g., the computing device described in FIG. 1), (2) as interconnected machine logic circuits or circuit modules (i.e., hardware) within the computing device and/or (3) a combination of software and hardware of the computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for effectively monitoring and analyzing glucose levels collected via continuous glucose monitoring to diagnose diabetes and related conditions, it will become evident to those skilled in the art that the implementations are not limited thereto, but are applicable for monitoring and analyzing levels of any metabolite and diagnosing any disease, condition, or state, including for purposes of treatment or further research.

As used herein, the terms “about” or “approximately” when referring to a measurable value such as an amount, a percentage, and the like, is meant to encompass variations of +20%, +10%, +5%, or +1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

It should be appreciated that the logical operations described herein with respect to the various figures may be implemented (1) as a sequence of computer implemented acts or program modules (i.e., software) running on a computing device (e.g., the computing device described in FIG. 1), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 1, an example computing device 100 upon which the methods described herein may be implemented is illustrated. It should be understood that the example computing device 100 is only one example of a suitable computing environment upon which the methods described herein may be implemented. Optionally, the computing device 100 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 100 typically includes at least one processing unit 106 and system memory 104. Depending on the exact configuration and type of computing device, system memory 104 may be volatile (such as random access memory (RAM)), non-volatile (such as read-only memory (ROM), flash memory, etc.), or some combination of the two. This most basic configuration is illustrated in FIG. 1 by box 102. The processing unit 106 may be a standard programmable processor that performs arithmetic and logic operations necessary for operation of the computing device 100. The computing device 100 may also include a bus or other communication mechanism for communicating information among various components of the computing device 100.

Computing device 100 may have additional features/functionality. For example, computing device 100 may include additional storage such as removable storage 108 and non-removable storage 110 including, but not limited to, magnetic or optical disks or tapes. Computing device 100 may also contain network connection(s) 116 that allow the device to communicate with other devices. Computing device 100 may also have input device(s) 114 such as a keyboard, mouse, touch screen, etc. Output device(s) 112 such as a display, speakers, printer, etc. may also be included. The additional devices may be connected to the bus in order to facilitate communication of data among the components of the computing device 100. All these devices are well known in the art and need not be discussed at length here.

The processing unit 106 may be configured to execute program code encoded in tangible, computer-readable media. Tangible, computer-readable media refers to any media that is capable of providing data that causes the computing device 100 (i.e., a machine) to operate in a particular fashion. Various computer-readable media may be utilized to provide instructions to the processing unit 106 for execution. Example tangible, computer-readable media may include, but is not limited to, volatile media, non-volatile media, removable media and non-removable media implemented in any method or technology for storage of information such as computer readable instructions, data structures, program modules or other data. System memory 104, removable storage 108, and non-removable storage 110 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 106 may execute program code stored in the system memory 104. For example, the bus may carry data to the system memory 104, from which the processing unit 106 receives and executes instructions. The data received by the system memory 104 may optionally be stored on the removable storage 108 or the non-removable storage 110 before or after execution by the processing unit 106.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

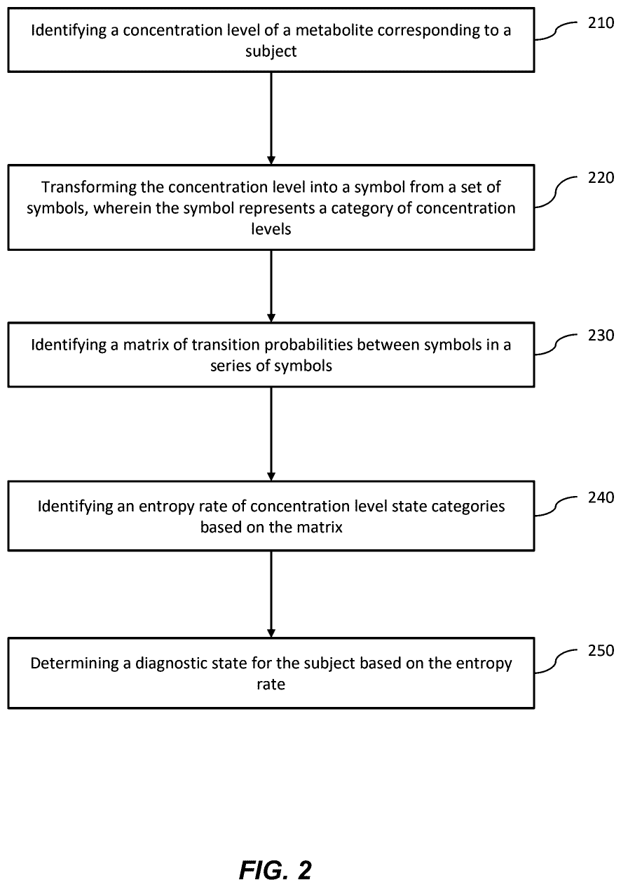

FIG. 2 is a flowchart of an example method for monitoring levels of a metabolite for use as diagnostic markers.

At 210, the method includes identifying a concentration level of a metabolite corresponding to a subject. A concentration level may include a blood concentration level, an interstitial concentration level, a sweat concentration level, a urine concentration level, or any other concentration level useful for identifying relevant diagnostic information in the subject. A metabolite may include glucose or any other metabolite, including any carbohydrate, fat, salt, drug, hormone, cell type, toxin, or potential toxin. In a preferred embodiment, the method includes identifying an interstitial glucose concentration level in a human subject.

The level may reflect a level measured in a sample taken from a subject or measured more directly from the subject. The metabolite may be a concentration level or any other measurement.

Data relating to a subject may be obtained using opt-in procedures, and may be anonymized or pseudo-anonymized to remove data that is not necessary for or helpful to the effective operation of the method. Data may be collected using any device that may be used to collect or monitor such data, including hospital devices for monitoring data, personal or portable devices capable of monitoring such data, or other computer devices (which may be used to enter data obtained through any other means). Data may further be obtained from other databases. Collected data may be described as a “trace,” such as a “CGM trace” for CGM data.

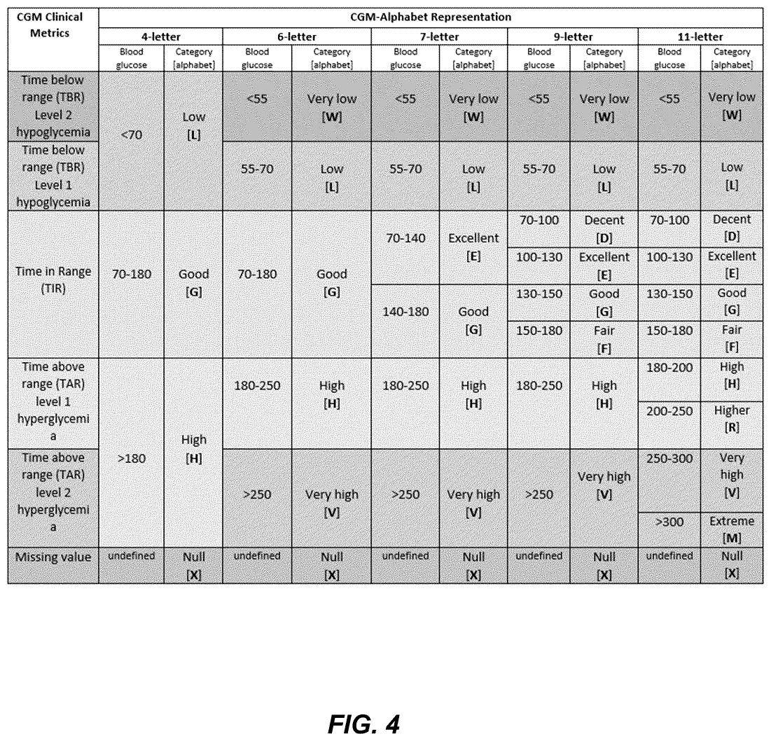

Then, at 220, the method includes transforming the concentration level into a symbol from a set of symbols, wherein the symbol represents a category of concentration levels. Transforming may include a simple mapping of a range or other category of concentration levels to particular symbols. Categories may include a range of values (with an upper and/or lower bound), a null category for invalid values or values not available, or other categories of like values. Mappings may include, for example, any of the mappings described at FIG. 4.

In alternative embodiments, transforming may include a more complex transformation, including a transformation determined by a more complex imperative program, including known techniques for sorting and categorization, or any machine learning technique useful for categorizing data, particularly if such a technique can be used efficiently for real-time categorization of concentration levels.

The set of symbols may be referred to an alphabet, but the symbols need not be letters, and may include numerals or any character that may be effective for encoding a concentration level. Alphabets of various sizes may be used to balance efficiency (wherein smaller alphabets may provide for more efficient analytics that distinguish between broad state categories) and accuracy (wherein larger alphabets may provide for more detailed analytics) or any of a variety of other factors, as may depend on the specific case at hand. Several exemplary embodiments include alphabet sizes of 4, 6, 7, 8, 9, and 11.

In some embodiments, a symbol, string of symbols, or the index of a symbol in a string may be associated with further data or metadata about the concentration level or associated state, including the concentration level itself, a time at which the concentration level was measured, or information about a subject, such as a diagnostic state of whether or not the subject has diabetes. Diagnostic states may be described, for example, as a positive result, negative result, or no data, or by a most recent test result and time.

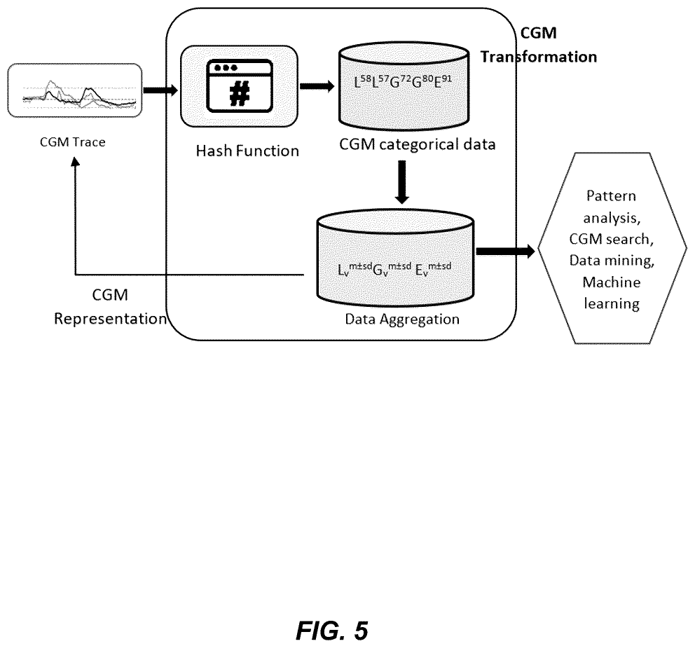

The method may further include identifying one or more strings of symbols, wherein each string of symbols represents a series of one or more symbols corresponding to a series of transformed concentration levels. For example, in an embodiment where a glucose concentration level of 90 units corresponds to an E symbol, a glucose concentration level of 68 units corresponds to an L symbol, and a glucose concentration level of 183 units corresponds to an H symbol, and a series of three successive glucose concentration readings are 90, 68, and 183 units, a corresponding string of transformed concentration levels representing that series of readings may be “ELH.” Strings may be any length of symbols, including any repeating pattern of symbols. Strings may include overlapping segments of a greater string; for example, if a string for a full day of measurements is “FGEFFEDFELDGHFHFFGDFHVFEXXEFGDE,” further strings may include “DFELD,” “LDGHFHFFGD,” and “GDFHVFEX.” Strings may further be compressed using any text compression method, particularly including a text compression method optimized to the size of the alphabet or to repetitive data. For example, a compressed text form of a string with five “F” symbols in a row, followed by 16 “P” symbols, followed by four “H” symbols, may be encoded as “F5P16H4.” Such compression is depicted in further detail at FIG. 5.

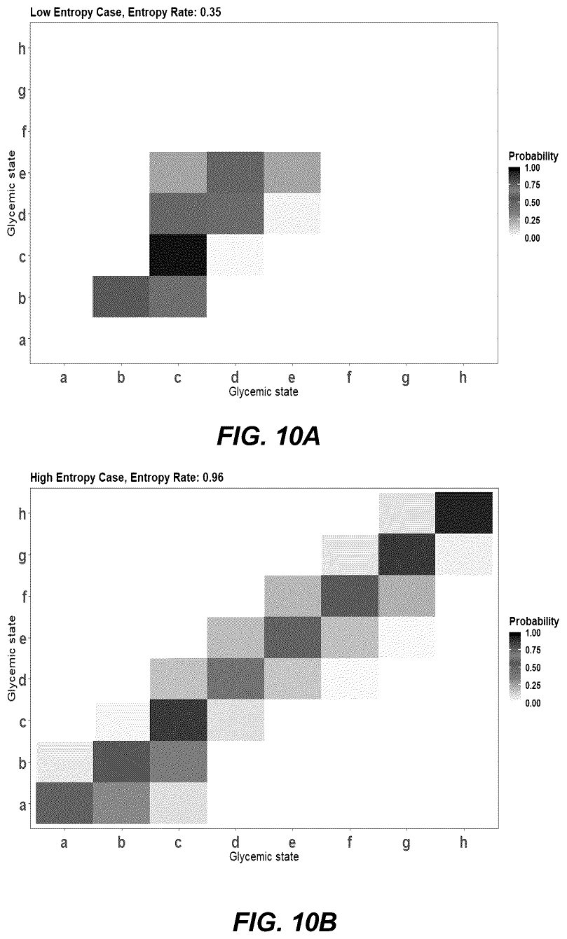

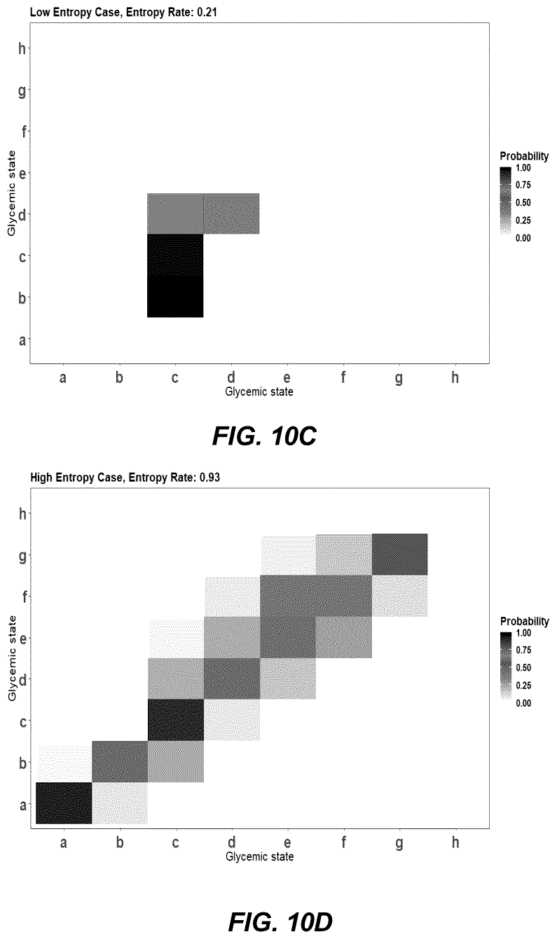

Next, at 230, the method includes identifying a matrix of transition probabilities between symbols in a series of symbols. The matrix may be referred to as a “transition probability matrix.” A transition probability matrix may be a computational representation of transition probabilities or a visualization of transition probabilities. A transition probability may be the probability that a state will transition to another state, wherein a state is a category of concentration levels represented by a symbol. Exemplary visualizations of transition probability matrices are depicted at FIGS. 10A-D.

Transition probabilities may be determined in a manner consistent with the Markov property, which is to say that a transition probability can be determined entirely based on the current state. Accordingly, the transition probability matrix may correspond to transitions between nodes in a Markov chain, where the Markov chain describes a long series of sequential states through a series of symbols.

A transition probability may be determined, for example, for a transition (c, d) representing a transition probability or historical frequency with which state d transitions to state c. A transition probability may be calculated using the historical frequencies of previously identified transitions alone or in combination with any other data or statistical method that may be useful in calculating transition probabilities. A full count of transitions (d, c) may be collapsed to a probability or relative frequency of transitions (d, c).

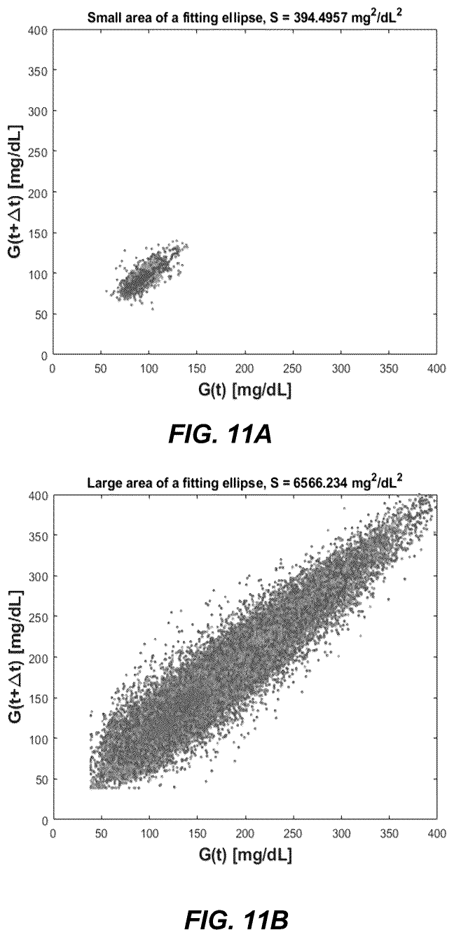

Transition probabilities may be visualized through a visualization of a transition probability matrix, as seen in FIGS. 10A-D. Concentration levels may be plotted on a Poincaré plot, such as the Poincaré plots described at 320 or visualized in FIGS. 11A-B, and may be correlated to transition probability matrices. For example, each visualization may be mapped to an equivalent scale, or one visualization may be created superimposing a transition probability matrix on top of a Poincaré plot.

Then, at 240, the method includes identifying an entropy rate of concentration level state categories based on the matrix. The entropy rate may be a measurement of entropy across transition probabilities. An entropy rate may be identified through calculations, computations, or other processing of the matrix.

The entropy rate may be a measurement may be a meta-measurement of entropy across transition probabilities, and may represent an overall rate of entropy, instability, or propensity of states to change into different states. Alternatively, the entropy rate may be framed as a stability rate, where a higher stability rate equates to a lower entropy rate, and vice-versa.

An entropy rate may be discovered through calculations, computations, or other processing of the matrix, including by a direct calculation of the probability that each state will change to a different state (or a probability that each state will lead to the state staying the same); or, as an alternative example, by use of a trained machine learning model that is trained to recognize Markov chains and determine an entropy rate. Probabilities may be framed as stability measurements or entropy measurements.

Next, at 250, the method includes determining a diagnostic state for the subject based on the entropy rate. Diagnostic states may include suggested or certain diagnoses, intermediate or final diagnoses, or any other type of diagnostic determination. Determining a diagnostic state may be an automated process; a manual process based on an output of the entropy rate or other data identified above; or a combined process that involves both automated and manual interpretation of data.

Diagnostic states may include suggested or certain diagnoses, intermediate or final diagnoses, or any other type of diagnostic determination. For example, a diagnostic state may include a determination of whether or not the subject has a particular condition, such as diabetes; a likelihood that a given diagnostic test will result in a positive or negative result; a state of progression of a disease, such as a stage of diabetes; a determination regarding the effectiveness of a treatment; a determination regarding a particular type, family, or category of a disease; a recommendation of next steps, including a recommended treatment or a recommendation of non-treatment; or any other output of recognized patterns or state-describing data for use by a medical professional in determining a diagnostic state.

Diagnostic states may be determined using the entropy rate alone or in combination with any other data, including, for example, other data about the subject, such as a medical history; data about other subjects, such as data used for comparison; a trained machine learning model, or a recommendation or initial impression from a medical professional or other user.

The term “user” here may refer to any medical professional, including, for example, a doctor, nurse, or paramedic; a professional in a medicine-adjacent profession, such as a hospital billing professional or an insurance company professional; a patient or subject, or the authorized representative of the patient or subject; a researcher; or any other user. Any data displayed or provided to a user may be anonymized or otherwise disposed as to protect the privacy of the subject of that data.

Determining a diagnostic state may be an automated process, such as a simple determination that an entropy rate of 40% or above indicates a positive diagnosis, and that an entropy rate below 40% indicates a negative diagnosis. As another example, determining a diagnostic state may be a process of artificial intelligence, such as a process of machine learning where a model is trained on prior diagnostic data and used to recognize a pattern of connections between entropy rates and diagnostic determinations.

In further embodiments, determining a diagnostic state may be a manual process based on an output of the entropy rate or other data identified above. For example, a program may present a medical professional with visualizations of the transition probability matrix and visualizations of a corresponding Poincaré plot, allowing the medical professional to determine a diagnostic state based on the visualizations.

In additional embodiments, determining a diagnostic state may be a combined process that involves both automated and manual interpretation of data. For example, a program may present a medical professional with a suggested diagnosis, a confidence rating based on the entropy rate, and a text explaining a reasoning behind the suggested diagnosis. Reasoning text may be pre-written, such as by a human, automatically generated, such as by an artificial intelligence or other natural language generation method, or generated through a combination of predetermined text and natural language processing, such as a simple imperative method that selects a pre-written text from several confidence categories (e.g., “high confidence, positive result;” “high confidence, negative result,” “medium confidence, positive result;” “medium confidence, negative result,” “low confidence, positive result;” “low confidence, negative result,” “no clear result”) and modifies the text based on specific details, including a precise entropy rate or stability rate, an explanation of how that entropy rate or stability rate was found, other data reflecting the processing history or underlying data about the subject, recommended next steps, or instructions for the medical professional to further utilize the application, such as to review other perspectives of the diagnostic state.

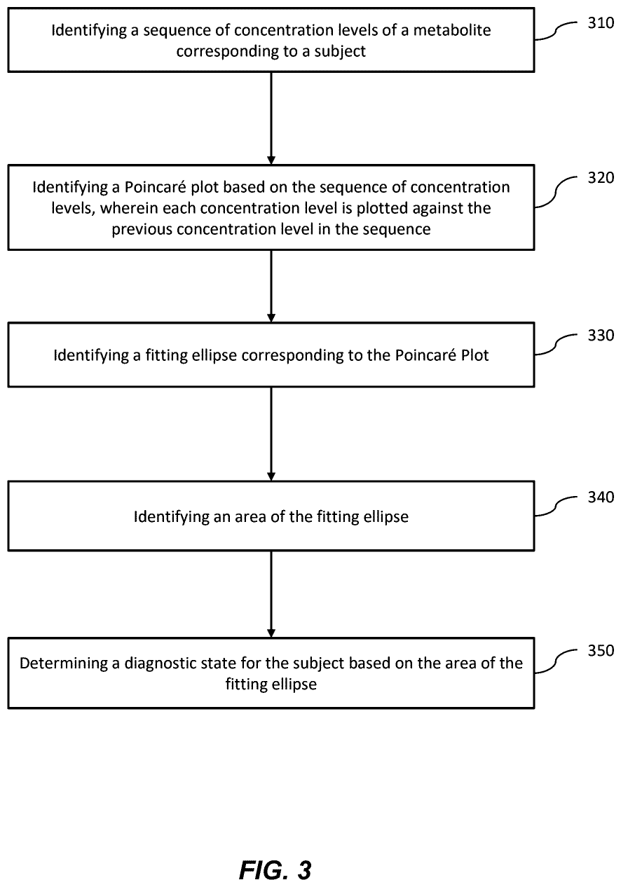

FIG. 3 is an exemplary flowchart for an alternate method of monitoring levels of a metabolite for use as diagnostic markers through use of Poincaré plot geometry.

At 310, the method includes identifying a sequence of concentration levels of a metabolite corresponding to a subject. Concentration levels may be identified as described at 210.

A sequence of concentration levels may be any ordered set, including a set of concentration levels identified at regular intervals, such as once every day or once every fifteen minutes, or alternatively at irregular intervals (for example, every hour at night, every fifteen minutes during the day, and every five minutes at mealtimes), according to a schedule, or in response to any condition (such as whenever a user or medical professional takes a measurement, or as triggered by any other event in a computational event system).

Then, at 320, the method includes identifying a Poincaré plot based on the sequence of concentration levels, wherein each concentration level is plotted against the previous concentration level in the sequence.

Concentration levels or transitions be plotted on a Poincaré plot, such as the Poincaré plots at FIG. 11A-B, either by individual values in the sequence of values or transformed values transformed according to the method at step 220. Individual values

A Poincaré plot may be represented as a visualization, or may be represented in purely mathematical terms such as a matrix, array, or object for processing by a computer program that interprets such data through methods of Poincaré plot geometry.

A visualization of a Poincaré plot may be output to a user, such as at 350.

Next, at 330, the method includes identifying a fitting ellipse corresponding to the Poincaré plot. The fitting ellipse may be identified through known statistical methods for fitting an ellipse to data of a Poincaré plot, scatter plot, or similar data or graph. A fitting ellipse may be identified in mathematical terms, drawn in a visual form, or both.

A fitting ellipse may be identified by mathematical means, including known statistical methods of identifying a fitting ellipse, such as by a least squares method, or through processing of a visual representation of a Poincaré plot, such as through visual recognition methods.

In further embodiments, identifying a fitting ellipse may include preparing a visualization of the fitting ellipse. The visualization may be placed on a chart with the same axes as the Poincaré plot, or superimposed on the Poincaré plot or a copy of the Poincaré plot. The visualization may be provided to a user, such as at 350.

Then, at 340, the method includes identifying an area of the fitting ellipse. The area may be calculated, computed, or determined by any other means.

The area of the fitting ellipse may be calculated or computed. For example, the area may be calculated by identifying the principal axes of the fitting ellipse, such as the minor axis and major axis on which minor radius 1202 and major radius 1204 lie, and performing the calculation:

$$Area = π r 1 r 2$$

where r1 represents minor radius 1202 and r2 represents major radius 1204.

In further embodiments, the area may be calculated by any other method of calculating the area of an ellipse, including by computer algorithms that calculate area by visual recognition or similar methods, or by a process of artificial intelligence, such as a machine learning model that is trained to identify and calculate areas of ellipses.

In alternate embodiments, identifying an area may include identifying a modified area, such as an area of a fitting ellipse plus another relevant area found in the Poincaré plot, or other factor relevant to the stability or diagnostic state of the subject.

Next, at 350, the method includes determining a diagnostic state for the subject based on the area of the fitting ellipse. A lower area may reflect a higher stability or lower entropy, and a higher area may reflect less stability or higher entropy.

Determining a diagnostic state may be performed in conjunction with any other methods, including the methods described at 250.

A diagnostic state may be determined using additional calculations, computations, or other processing of the Poincaré plot, including by a direct calculation of the probability that each state will change to a different state (or a probability that each state will lead to the state staying the same); or, as an alternative example, by use of a trained machine learning model that is trained to recognize visual data such as a visualization of an ellipse.

The method may also include outputting the diagnostic state, including any data related the diagnostic state, the fitting ellipse, or the Poincaré plot. Outputting the data may be performed using any method described at 250.

For example, a program may present a medical professional with visualizations of the Poincaré plot, including a visualization that presents the area of a fitting ellipse in the Poincaré plot visually, and allows the medical professional to determine a diagnostic state based on the visualizations.

FIG. 4 is an exemplary table of potential alphabets representing different categories of data for continuous glucose monitoring. Particularly, FIG. 4 portrays potential 4-letter, 6-letter, 7-letter, 9-letter, and 11-letter alphabets; however, any size of alphabet may be used to correspond to that number of categories. In a preferred embodiment, an 8-symbol alphabet may be used. FIG. 4 portrays ranges that correspond to categories that are thought to be useful in continuous glucose monitoring, but any range may be used, as may correspond to measurements of any metabolite.

FIG. 5 depicts an exemplary process for dynamic continuous glucose monitoring (CGM) and indexing of states for diagnostic purposes.

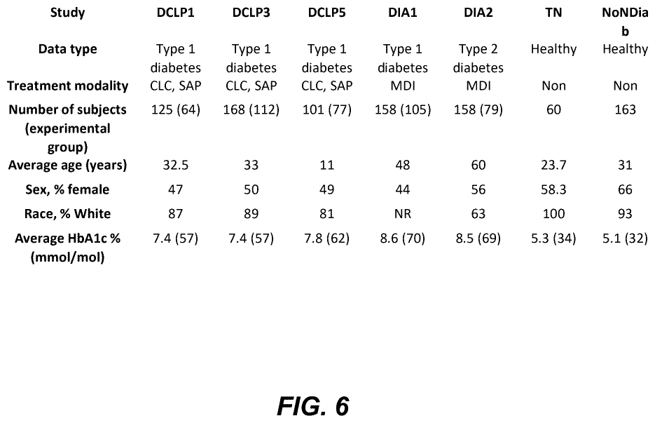

FIG. 6 is a table reflecting clinical and demographic characteristics of seven different data sets used for analysis. Abbreviations: CLC, closed-loop control; HbA1c, glycated hemoglobin; MDI, multiple daily injections; NR, not reported; SAP, sensor-augmented pump.

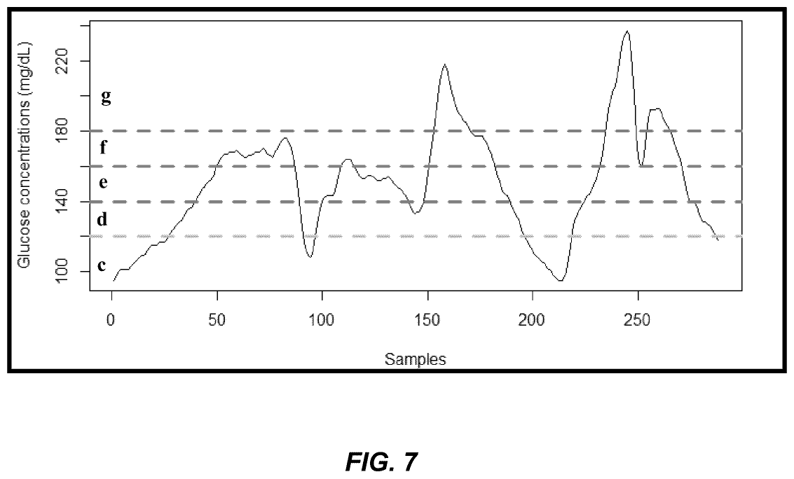

FIG. 7 is an explanatory example of encoding a daily CGM profile. The symbolic representation for this CGM daily profile can be represented as a string series: “cccccccccddddeeefffffffffffffdccdeeeffeeeeeeeeeddefggggggffffeeddccccccccddeefgggggffgggff eddddd” with 8 symbols, and a 96-word size.

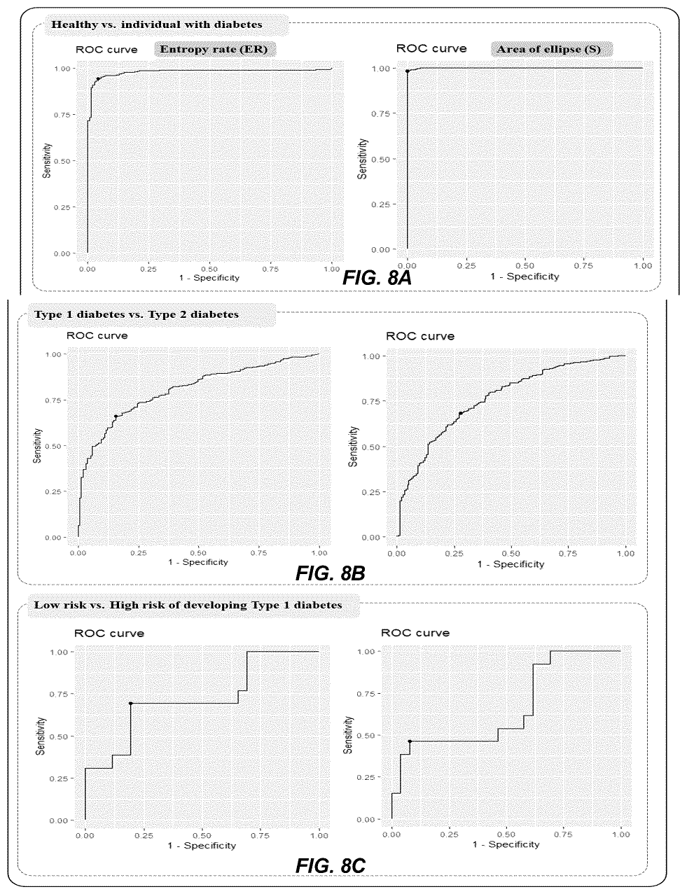

FIGS. 8A-C portray exemplary ROC curves for three different cases of discrimination to evaluate the discriminative power of the ER and S as biomarkers. FIG. 8A presents ROC curves for the discrimination between healthy and individuals with diabetes. FIG. 8B presents ROC curves for the discrimination between type 1 diabetes and type 2 diabetes individuals. FIG. 8C presents ROC curves for the discrimination between individuals with low risk and high risk of developing type 1 diabetes.

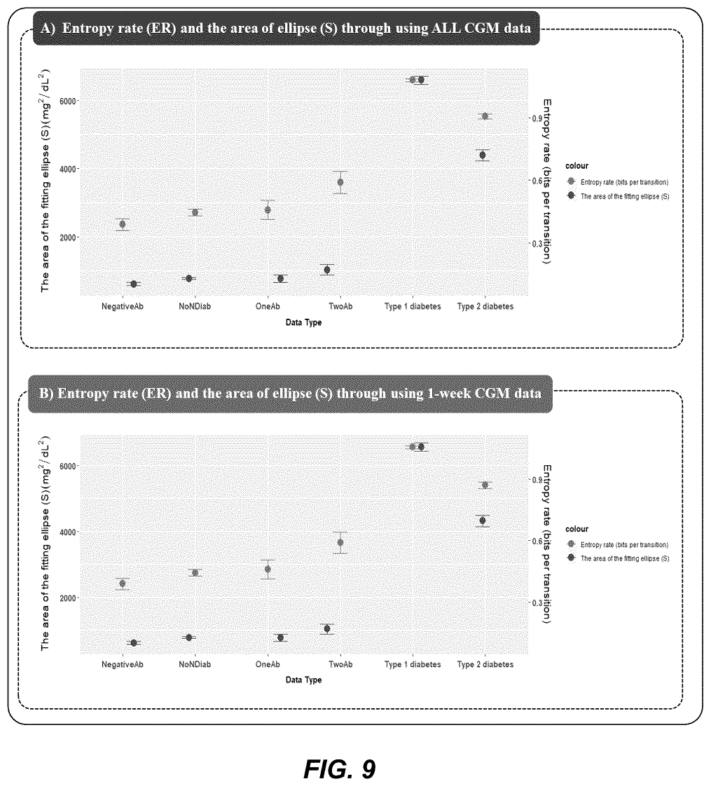

FIG. 9 presents scatterplots with a standard error for the entropy rate criterion (bits per transition), and the area of a fitting ellipse (mg2/dL2) for the different datasets (TN study: NegativeAb, OneAb, and TwoAb (≥2 Ab), NoNDiab study, Type 1 diabetes studies with different treatments, and Type 2 diabetes study with multiple daily insulin injections (DIA2)). Under plot A, the entropy rate criterion and the area of a fitting ellipse through using ALL CGM data; under plot B, the entropy rate criterion and the area of a fitting ellipse through using 1-week CGM data. Abbreviations: Islet Autoantibody (Ab).

FIGS. 10A-D presents an illustrative example of a transition probability matrix for four different subjects. FIG. 10A may represent, for example, a “healthy” individual (low entropy); FIG. 10B may represent a diabetic individual (high entropy); FIG. 10C may represent an individual who tests negative for an autoantibody (low entropy); and FIG. 10D may represent an individual who tests positive for an autoantibody (high entropy). These figures may portray any biomarkers or be used for any type of diagnosis.

FIGS. 11A-B present Poincaré plots for two subjects. FIG. 11A may represent the same measurement of the same subject as FIG. 10A; FIG. 11B may similarly correspond to FIG. 10B. Accordingly, FIG. 11A may represent, for example, a “healthy” individual (small fitting ellipse; low entropy) and FIG. 11B may represent a diabetic individual (large fitting ellipse; high entropy). These figures may portray any biomarkers or be used for any type of diagnosis.

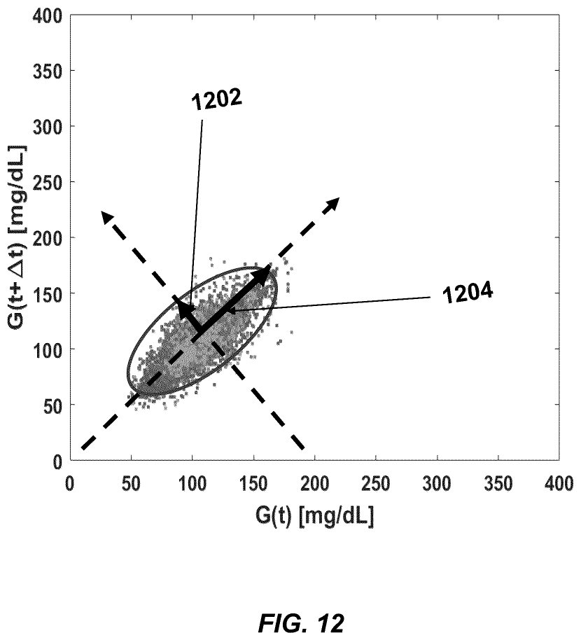

FIG. 12 presents an illustrative example of a Poincaré plot, a fitting ellipse of the Poincaré plot, and line segments useful for calculating the area of the fitting ellipse. Line segment 1202 may represent a minor radius or width of the ellipse, and may be referred to elsewhere as “SD1;” line segment 1204 may represent a major radius or length of the ellipse, and may be referred to elsewhere as “SD2.”

Examples herein are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Two different dynamical-system methods are proposed in this invention: The first method is based on the entropy of the dynamical system representing diabetes, and consists of four steps: (i) Encoding Continuous Glucose Monitoring (CGM) data into strings of letters, each letter identifying a specific Glycemic State; (ii) Computing a transition probability matrix between sequential daily Glycemic States; (iii) Computing the proportion of time an individual spends in each Glycemic State, i.e., the stationary distribution of the transition probability matrix, and (iv) Computing the entropy rate of the encoded CGM process using the transition probability matrix and stationary distribution for each individual. With these steps are accomplished, any daily CGM profile is mapped to a specific string of letters, and the string series (symbolic representation) for an individual, which is then used as a marker of disease progression or treatment effectiveness. The transition probability matrix of this process is used to augment the information traditionally provided by the Ambulatory Glucose Profile (AGP) and the associated times in ranges of the original daily CGM profile. In addition to AGP and its associated metrics, the transition probability matrix and the entropy rate criterion provide information about the timing and the inter-day variability of the clinically-relevant glycemic events of a person, which represents the progression of diabetes of this individual over time.

The second method is based on visualizing and quantifying the attractor of the dynamical system representing diabetes, and consists of two steps: (i) Representing CGM data through Poincaré plot (PP) as a visual technique for analyzing dynamic systems, and (ii) Computing the area of a fitting ellipse(S) through the descriptors of PP Geometry.

Potential applications of the Two Methods include: (i) Data structuring and dimensionality reduction; (ii) Encoding of daily CGM profiles; (iii) Distinguishing between healthy and diabetic individuals, as well as between different degrees of impaired glucose tolerance (e.g. pre-diabetes, gestational diabetes); (iv) CGM replacement for common diabetes diagnostic tests; (v) CGM pattern recognition and forecast; (vi) Tracking disease progression or treatment effectiveness, and (vii) Differentiating stages of diabetes.

The major advantage of this invention is the ability to be used for screening and diagnosis of diabetes, identify those with a heightened risk of progression to diabetes, and describe observing disease or treatment progression, all of which is done using 1-2 weeks of minimally invasive CGM at home. These procedures are much less invasive, demanding, and expensive, compared to the current clinical standard of Glucose Tolerance tests that can be done only in inpatient conditions.

This invention generally relates to medicine and medical devices, as used for tracking the progression of diabetes mellitus over time, screening or prediction of disease progression in at-risk individuals, and insulin treatment of diabetes and other metabolic disorders, including but not limited to pre-diabetes, impaired glucose tolerance, type 1 and type 2 diabetes (T1D, T2D), latent autoimmune diabetes in adults (LADA), or gestational diabetes. In alternative embodiments, the invention defines new mathematical/dynamical markers, the first one computed from the transition probability matrix of a sequence of daily CGM profiles, which describes the evolution of an individual across a predefined set of glycemic states. One method of the invention includes encoding CGM data into strings of letters and, therefore, reducing the dimensionality of the daily CGM profiles, preserving critical clinically-relevant characteristics of the daily CGM profile. Then, the transition probability matrix of the converted daily CGM profiles is computed by using predefined glycemic states, defined as different glycemic intervals. The proportion of time an individual spends in each glycemic state is computed by a stationary distribution, and finally, the entropy of this process is computed. The Transition Probability Matrix and the Entropy Rate (ER) of each individual can then be used as a novel criterion of diabetes diagnosis and tracking the progression of diabetes mellitus over time, augmenting the information traditionally derived from the Ambulatory Glucose Profile (AGP) and its associated times in ranges with new markers reflecting the volatility of glycemic control, which often shows features of disturbance prior to the onset of hyperglycemia—the current defining characteristic of diabetes. Another method of the invention derives a second marker computed from the area of a fitting of the Poincaré plot (PP) Ellipse(S) for describing the dynamics of CGM data. A smaller, more concentrated plot indicates system (healthy individual) stability, whereas a more widespread PP indicates system (diabetic individual) irregularity, reflecting in our case poorer glucose control (unstable diabetes) or progression to the onset of diabetes. The major advantage of this invention is the ability to use new indices (mathematically and visualization) for screening and early diagnosis of diabetes, identifying those at a heightened risk of progression to diabetes, and quantifying disease progression or treatment success/failure for those who have already been diagnosed.

Diabetes represents one of the considerable health challenges of the 21st century. According to International Diabetes Federation (IDF) Diabetes Atlas 2021, 537 million adults have diabetes worldwide in 2021, and this figure is projected to touch at least 783 million by 2045. Globally, almost one-in-two adults of age ≥20 years old (44.7%; around 240 million) are living with undiagnosed diabetes. Although people with undiagnosed diabetes do not have as much hyperglycemia as people with established diabetes, they still have high risk for diabetes complications. Therefore, it is crucial for people with diabetes to be diagnosed as early as possible to prevent or delay long-term health complications, improve quality of life, avoid premature death, and reduce distress. The American Diabetes Association (ADA), the International Expert Committee (IEC), and the World Health Organization (WHO) have recommended different diagnostic criteria for diabetes. These criteria are based on procedures requiring blood draws, such as plasma glucose criteria, either the fasting plasma glucose (FPG) level or the 2-hour plasma glucose (2hPG) level after a 75-g oral glucose tolerance test (OGTT), or glycosylated hemoglobin (HbA1c) level, with different cut-point for diagnosing diabetes. Diabetes is defined with cut-point of an FPG level ≥126 mg/dL (7.0 mmol/L), and a 2hPG level ≥200 mg/dL (11.1 mmol/L), with different ranges for impaired fasting glucose criterion (100 to 125 mg/dL by the ADA vs. 110 to 125 mg/dL by the WHO). Diabetes is also defined with an HbA1c level ≥6.5% (48 mmol/mol) by the ADA and the IEC, with different ranges for prediabetes (5.7% to 6.4% by the ADA vs. 6.0% to 6.4% by the IEC).

As a test for the initial diagnosis of diabetes, FPG is easier than the 2hPG to perform at the hospital but requires fasting overnight before a blood draw too, which is a time-consuming process. However, the 2hPG is more sensitive than FPG alone and could capture the dynamic of glucose spikes after the meal, but is limited by poor reproducibility in the 60%-80% range. Besides that, the macronutrient content of the meal before the OGTT may affect the results. In addition, OGTT-defined categories have limitations, especially when one considers the transition from dysglycemia to the onset of the disease. The HbA1c is an accurate and reproducible test that has the advantage of convenience compared with FPG or 2hPG, because it does not require fasting overnight. However, HbA1c may be an unreliable test under certain conditions such as hemolytic anemia, iron deficiency, hemoglobinopathies, and pregnancy. For example, HbA1c is not particularly useful for detection of gestational diabetes because the process of hemoglobin glycation takes 2-3 months, which is too slow for detecting the onset of diabetes during pregnancy. Furthermore, the racial differences in HbA1c independent of blood glucose have been reported in many different studies.

In the last two decades, continuous glucose monitoring (CGM) systems have proved the ability to track dynamic glycemic fluctuations, trends, and patterns over time allowing for the optimization of medical therapy, minimizing the risk of hypoglycemia, improving the quality of life, screening, or diagnosing diabetes. Several studies have demonstrated that CGM-derived time-in-range values (TIR, 70-180 mg/dL) have a high correlation with HbA1c, and could be used as a marker to predict diabetes-related complications. Over the years, a number of CGM-derived glycemic metrics were developed to assess the quality of glycemic control through aggregating CGM data to communicate a meaningful clinical message. However, representing CGM data through the Ambulatory Glucose Profile (AGP) and CGM-derived time in different ranges does not reflect the dynamics of blood glucose and the subtle gradual deterioration of glycemic control over time. Since the essential advantage of CGM is measuring time series of glucose values and capturing the process of glycemic control as it evolves, adding a temporal component to CGM-derived time-in-ranges becomes useful, for catching glycemic deviations early and tracking diabetes progression over time.

Recently, in several studies in adolescents and children, CGM data was used for detecting early hyperglycemia and to characterize participants who progressed to stage 3 type 1 diabetes with respect to the percent time they spent above 140 mg/dL. In addition, many studies have shown that integrating CGM data into machine learning models could help to develop predictive models that could assist clinicians in improving the screening, diagnosing, and treatment of diabetes. Recently, classifying and clustering daily CGM profiles into one of 32 sets of clinically similar clusters (CSCs) has been proposed, to allow tracking of daily glycemic changes over time.

Given the limitations of the current criteria and diagnostic tests for diabetes, in this invention, we propose new dynamical approaches that include two different methods. The first method is based on the entropy of the dynamical system representing diabetes, and includes: (i) Encoding CGM data into strings of letters, each letter identifying a specific Glycemic State; (ii) Computing a transition probability matrix between sequential daily Glycemic States; (iii) Computing the proportion of time an individual spends in each Glycemic State, i.e., the stationary distribution of the transition probability matrix, and (iv) Computing the entropy rate of the encoded CGM process using the transition probability matrix and stationary distribution for each individual. This method is based on the theory of semi-Markov chains, used here to describe the evolution of an individual across the predefined set of glycemic states, characterized by random duration of time spent in each state. The second method is based on visualizing and quantifying the attractor of the dynamical system representing diabetes, and includes: (i) Representing CGM data through Poincaré plot (PP) as a simple visual technique for analyzing dynamic systems, and (ii) Computing the area of a fitting ellipse(S) through the descriptors of PP Geometry.

In order to encode CGM data space into strings of letters, two steps were applied. The first step is to employ dimensionality reduction for the complete daily CGM profiles, where daily CGM traces of arbitrary length n (commonly n=288 data points per day) is converted to a string with length w, (w<n, typically w<<n). Therefore, the converted daily CGM traces can be represented in a w-dimensional space instead of an n-dimensional space. In this case, the CGM traces are divided into segments w of equal size S̅=S̅1̅, S̅2̅, . . . , S̅_̅{̅w̅}̅ where each time segment is summarized with the mean value of glucose data points that it includes. The i^{th} element of each time segment S̅ is calculated from the following equation.

$${\overline{S}}_{i} = \frac{w}{n} {\sum }_{j = \frac{n}{w} ( i - 1 ) + 1}^{( \frac{n}{w} ) i} {S}_{j} ,$$

where i ranges from 1 to w, Sⱼ is the jth element of the daily CGM traces, and a constant integer n/w is called the time segment size. Each segment represents a letter. In this way, the number of segments contained in daily CGM traces represents the number of letters of a word (or symbol). As a result, historical data are represented by “words”, each word corresponding to a daily CGM profile. In this implementation we use a number of segments w=96; however other approaches, e.g. w=24, or w=48 and reasonable as well. Therefore, the time segment size will be equal to 288/96=3. Such a time segment size (3 CGM data points equal to 15 minutes CGM time series length) ensures that key clinically relevant characteristics of the daily CGM profile are preserved, and the ability to observe the transition from one glycemic state to another per individual and study.

The second step deals with the definition of the letters through the limited alphabet size. The alphabet size is also an arbitrary integer a, where a>2 (e.g., for the alphabet={a, b, c}, a=3). Each letter is defined by a boundary of an interval known as breakpoints. In this implementation, the breakpoints define the following CGM glucose intervals (8 symbols/glycemic intervals): a (time spent <54 mg/dL), b (time spent 54-69 mg/dL), c (time spent 70-120 mg/dL), d (time spent 121-140 mg/dL), e (time spent 141-160 mg/dL), f (time spent 161-180 mg/dL), g (time spent 181-250 mg/dL), and h (time spent >250 mg/dL). In other words, if the segment mean is below the smallest breakpoint (<54 mg/dL), it is mapped to the alphabet symbol “a”, and if the segment mean is above the biggest breakpoint (>250 mg/dL), it is mapped to the alphabet symbol “h” and so on. Other sets of breakpoints defining a different alphabet are feasible as well.

In order to compute the transition probability matrix for each daily CGM profile, eight predefined Glycemic States m={a, b, c, d, e, f, g, h} were used as described above. In this embodiment, we use m=8 states, but m could be any other not-too-large number. Transition or change in the daily CGM traces from one state to another in the next period of time (e.g., transition from TIR to time above range TAR), is a random process and is expressed in the form of probability, and it is called the “transition probability” utilized to determine the probability of the next state. Each daily CGM profile can be presented by a transition probability matrix P=(Pᵢⱼ) of size m×m such that the individual entries per individual are the probability of moving from state i to state j. For every i and j, i,j 1, . . . , m, the P matrix should satisfy the following conditions: Pᵢⱼ≥0, ∀ i, j, and

$${\sum }_{j = 1}^{m} {P}_{i j} = 1 ,$$

∀ i where ∀ means “for every.”

The stationary distribution (a probability distribution that does not change over time) for the matrix WP is a vector, ψ, such that ψ WP=v, where ψⱼ≥0, for all j and Σ_{jϵm}ψⱼ=1. Such a distribution is called stationary as ψ(WP)²=(ψWP)WP=ψWP=ψ. Therefore, the proportion of time the individual spends in a glycemic state j is approximately ψⱼ for all j, regardless of what the starting glycemic state was. Each individual has a specific vector ψ, representing the proportion of time spent this individual spends in the predefined Glycemic States m.

The entropy rate (ER) is defined as a measure of the uncertainty and complexity of daily CGM traces, where a smaller value of the ER corresponds to a more predictable and less complex daily CGM profile. Once we have computed the WP matrix and the proportion of time spent in the eight-glycemic states m per individual (I) through ψ, we can compute the ER per individual as follows:

$$ER ( I ) = - {\sum }_{i , j} {ψ}_{i} {WP}_{ij} {log}_{2} {WP}_{ij} .$$

The Poincaré plot (PP), or lag plot, was developed by a French mathematician Henri Poincaré in 1890, as a simple visual technique for analyzing dynamic systems (i.e., a classical approach to the visualization of nonlinear system dynamics-dynamics of CGM). The importance of this plot is that it is the two-dimensional (2-D) reconstructed glucose interval phase-space—the projection of the reconstructed attractor that describes the dynamics of the CGM time series. Typically, in CGM applications, the plot appears as a stretched cloud of points oriented along the line of identity, each point of the plot has coordinates G(t) on the x-axis and G(t+Δt) on the y-axis. Where G(t) is the glucose level at time t and G(t+Δt) is the glucose level at time t+Δt. Thus, the difference (y-x) coordinates of each data point represents the glucose rate of change occurring between times t and t+Δt. We constructed PPs with fixed Δt values of 15 min (previous studies have demonstrated that the optimal evaluation of the blood glucose rate of change would be achieved over periods of 15 min). A smaller, more concentrated plot indicates system (healthy individual) stability, whereas a more widespread PP indicates system (diabetic individual) irregularity, reflecting in our case poorer glucose control (unstable diabetes) or progression to the onset of diabetes. The time interval of 15 minutes was chosen to optimally represent the rate of CGM data change; however, other values are possible, ranging from 5 minutes to an hour.

The PP is characterized by several descriptors; SD1 and SD2 are two standard PP descriptors. SD1 (the width of the ellipse) is the SD of projection of the PP on the line perpendicular to the line of identity (y=−x; reflects the short-term variability), and SD2 (the length of the ellipse) is defined as the SD of the projection of the PP on the line of identity (y=x; reflects the long-term variability). Both descriptors are defined as:

$$SD {1}^{2} = \frac{1}{2} SD Δ {G}^{2} ,$$

$$SD {2}^{2} = 2 {SD}^{2} - SD {1}^{2} .$$

Where, SDΔG is the SD of the successive (G_{t+Δt}−Gₜ) intervals, and SD is the standard deviation of the glucose levels. Additionally, we could compute the area of a fitting ellipse(S) which reflects the total variability as:

$$S = π \cdot SD 1 \cdot SD 2$$

The data used to illustrate the method were collected across seven different studies (FIG. 6), which tested various treatment modalities in individuals with type 1 diabetes or type 2 diabetes, besides healthy individuals without and with a different islet autoantibody (Ab) status (Negative Ab, 1 Ab, 2 or more Ab). CGM data were collected for the entire duration of the studies. A summary of the study protocols is outlined hereunder:
I. The International Diabetes Closed Loop Trial (iDCL)-Protocol 1 (DCLP1; NCT02985866): a 3-month, multi-center, parallel-group, randomized trial where 125 adults with type 1 diabetes were assigned to a mobile closed-loop control (CLC) system or a sensor-augmented pump (SAP);
II. iDCL-Protocol 3 (DCLP3; NCT03563313): a 6-month, multi-center, randomized trial where 168 adults with type 1 diabetes were assigned in a 2:1 ratio to receive treatment with a CLC system or SAP;
III. iDCL-Protocol 5 (DCLP5; NCT03844789): a 16-week, multi-center, parallel-group, randomized trial where 101 children 6-13 years of age with type 1 diabetes were assigned in a 3:1 ratio to CLC or SAP;
IV. Multiple daily insulin injections (MDI) and CGM in Diabetes (DIaMonD) in type 1 diabetes (DIA1; NCT02282397): a 24-week, multi-center, randomized trial where 158 adults 26-73 years of age with type 1 diabetes using MDI, were assigned in a 2:1 ratio to CGM or home blood glucose self-monitoring with intermittent blinded CGM;
V. DIaMonD in type 2 diabetes (DIA2; NCT02282397): a 24-week, multi-center, randomized trial where 158 adults 35-79 years of age with type 2 diabetes using MDI, were assigned in a 1:1 ratio to CGM or home BG self-monitoring with intermittent blinded CGM;
VI. TrialNet ancillary study in healthy participants (TN; NCT02663661): a 1-week CGM for 60 healthy relatives of people with type 1 diabetes with average age of 23.7 years, with zero (N=21), one (N=18), and ≥2 (N=21) autoantibodies, recruited from participants with known autoantibody status in the TrialNet Pathway to Prevention study;
VII. CGM in Healthy Nondiabetic Participants (NoNDiab; Type 1 diabetes Exchange Clinic Network Studies): CGM up to 10 days, multicenter, prospective study, a total of 163 nonpregnant, healthy, nondiabetic children and adults (age >6 years), with normal-weight have participated in this study.

Complete daily CGM profiles (e.g., a time series of 288 glucose data points collected every 5 min during the midnight-to-midnight 24 h period) from the 7 studies detailed above were used in the analysis. Individuals with a number of daily CGM profiles <70% of active CGM (at least has 5 complete daily CGM profiles) were excluded from the analysis. The entire set of daily CGM profiles for an individual were tested versus one week daily CGM profiles, to assess a minimum number of daily CGM profiles that could be used as a criterion/biomarker for diagnosing diabetes or predicting the early stages of preclinical type 1 diabetes.

Method 1: Entropy Rate of the dynamical system: Eight different glycemic states m were used as described above, in order to define the P matrix for each daily CGM profile per individual across the different studies. The weighted average transition probability matrix (WP) was computed for n daily CGM traces per individual through the number of transitions from state to another. Low ER transition matrix corresponds to a daily CGM profile for an individual with a high probability of transitions to the same state (e.g., spending significant time in “healthy” state c in a specific daily CGM profile with Pᵢᵢ=0.96), and medium or high ER transition matrix indicated increase volatility of the CGM trace.

Method 2: Dynamical System Attractor/Poincaré Plot: A smaller area of a fitting ellipse(S) indicates system stability, CGM traces for healthy individuals, or CGM traces for Negative Ab individuals. Whereas a bigger S indicates system irregularity, poorer glucose control (unstable diabetes), onset or progression of diabetes, or treatment efficacy in keeping glycemic excursions in check.

All daily CGM profiles are reduced to a symbolic representation and a WP matrix or one number (ER). In addition, they could be reduced to a 2-D simple visualization (PP), or one number(S) that can be used as input to decision support, clinical and automated treatment algorithms.

Any daily CGM traces can be represented in a 96-dimensional space instead of a 288-dimensional space, and each daily CGM profile can be represented as a string series (symbolic representation) with 8 symbols, and a 96-word size (see FIG. 7). As noted, this could be further reduced to 48 and even 24 dimensions, depending on the tolerance of approximating CGM data volatility.

One application of the methods is the ability to distinguish between degrees of pre-diabetes, impaired glucose tolerance, T1D, T2D, LADA, or gestational diabetes. For example, FIG. 8 shows the ROC curve for the three different cases of discrimination, where the ER with an AUC of 0.98 (95% CI: 0.97-0.99), and S with an AUC of 0.99 (95% CI: 0.99-1.00), were able to discriminate individuals with diabetes from healthy with 0.7552 (bits per transition), and 1993.911 (mg²/dL²) as the “optimal” cut-off, having 0.94%, 0.98% sensitivity and 0.96%, 1.00% specificity, respectively (FIG. 2A).

FIG. 8B shows the ROC curve with an AUC of 0.81 (95% CI, 0.77-0.84), and AUC of 0.76 (95% CI, 0.72-0.81), in which ER and S were able to discriminate individuals with type 2 diabetes from type 1 diabetes with 1.0069 (bits per transition), and 5112.975 (mg²/dL²), as the “optimal” cut-off, having 0.66%, 0.68% sensitivity and 0.85%, 0.72% specificity, respectively.

FIG. 8C shows the ROC curve with an AUC of 0.72 (95% CI, 0.58-0.86), an AUC of 0.66 (95% CI, 0.47-0.86), in which ER and S were able to separate individuals with low risk (Negative Ab and One Ab) from high risk of rapid progression to stage 3 type 1 diabetes (≥2 Ab), with 0.5225 (bits per transition), 923.645 (mg²/dL²), as an “optimal” cut-offs, having 0.69% sensitivity, 0.74% specificity, 0.46% sensitivity and 0.92% specificity, respectively.

Measuring fasting glucose level, A1C test, homeostatic (HOMA) assessment of insulin sensitivity and beta cell function, autoantibodies test, and oral glucose tolerance test (OGTT) are common clinical methods for the evaluation of the glycemic health state of a person and an early diagnosis diabetes tools. These, and other, common glycemic function tests typically require a physician visit, blood draws, and laboratory analysis. In the case of OGTT, several hours of testing are needed in a clinical setting. While cumbersome, these tests are required routinely in many situations, e.g. frequent OGTT in gestational diabetes. Moreover, OGTT-defined categories have limitations, especially when one considers the transition from “dysglycemia” to the onset of the disease.

The ability of the ER, transition probability matrix, and S to distinguish with high fidelity between healthy and diabetic individuals could serve as a replacement for these clinical tests-a 1-week CGM wear in a home environment, accompanied by a predefined schedule of meals and physical activity, will achieve diagnostic results similar to those accepted in the clinical practice, while greatly simplifying the data collection (see FIGS. 9-11).

The transition probability matrix describing the evolution of a patient across the predefined glycemic states, is a natural tool for observation of disease or treatment progression. Pattern recognition, or recurrent behaviors, are reflected by patterns, or cycles, detected in the transition probabilities from one state to the next. Short- or long-term forecasts of glycemic control are based on probability patterns or recurrent visits to a certain subspace of the Markov chain state space. The latter is a subject of the theory of semi-Markov chains, which result from aggregation of the state space into relevant subsets, characterized by random duration of time spent in each subset. In addition, a smaller S indicates system stability, whereas a bigger S indicates system irregularity, poorer glucose control (unstable diabetes), or progression to the onset of diabetes.

Disease deterioration or progression is signified by transitions into undesired states and, conversely, a successful treatment optimization of medication titration is reflected by transition into clinically desirable states. In a practical application, the state space of the Markov chain is defined to correspond to the transition probability matrix and the ER defined by this invention. In addition, an increase in the area of the ellipse(S) could be a sign of increased variability and progression to stage 3 T1D (dysglycemia), or bad glycemic control.

One application of the methods of this invention is the ability to identify individuals who may progress to the onset of diabetes (see FIG. 8C above). FIG. 14 shows an example of the transition probability matrix for an individual who is Negative for type 1 diabetes autoantibodies (e.g., at low risk for T1D), characterized by a low entropy rate and a small area of the PP ellipse vs. the transition probability matrix of an individual with 2 or more autoantibodies (e.g., at high risk for T1D), characterized by a high entropy rate and a larger area of the PP ellipse.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computer-implemented method of analyzing a set of glucose concentration levels with data from continuous glucose monitoring (CGM), the method comprising:
using a computer with a processor connected to computer memory to implement software having computer implemented instructions performing a method comprising:
selecting a plurality of subsets of CGM data measurements from the set of glucose concentration levels;
accessing, from the computer memory, respective ranges of glucose concentration levels, wherein the respective ranges identify respective glycemic states;
assigning the respective subsets to one of the respective glycemic states and saving the respective glycemic states in the computer memory;
calculating a transition probability matrix comprising matrix entries corresponding to respective probabilities that a first respective glycemic state switches to a different respective glycemic state over a period of time;
calculating a stationary distribution for the transition probability matrix, wherein the stationary distribution represents a proportion of time spent in each of the respective glycemic states; and
calculating an entropy rate of the set of glucose concentration levels with the transition probability matrix and the stationary distribution;
determining a diagnostic state for the individual based on the entropy rate.

2. The computer-implemented method of claim 1, further comprising:
calculating a mean value for each of the subsets;
fitting the mean value into a respective range of the respective glycemic states; and
identifying the respective glycemic states with a symbol.

3. The computer-implemented method of claim 2, further comprising identifying the respective glycemic states with a symbol comprising a letter or a number.

4. The computer-implemented method of claim 1, further comprising performing the method with data from multiple CGM traces for an individual.

5. The computer-implemented method of claim 4, further comprising calculating a weighted average transition probability matrix (WTP) from respective transition probability matrices calculated from the CGM traces for the individual.

6. The computer-implemented method of claim 5, further comprising calculating the stationary distribution from the weighted average transition probability matrix.

7. The computer-implemented method of claim 6, further comprising calculating the entropy rate (ER(I)) for an individual with a formula comprising:
ER(I)=−Σ_{i,j} ψᵢ WTPᵢⱼ log₂ WTPᵢⱼ, wherein ψᵢ is the stationary distribution for the individual and WTPij is the weighted average transition probability matrix for the individual.

8. The computer-implemented method of claim 4, further comprising calculating Poincare′ plot (PP) descriptors for the respective CGM traces, wherein the Poincare′ plot descriptors comprise standard deviation (SD) calculations for the data from the respective CGM traces.

9. The computer-implemented method of claim 8, further comprising:
calculating a first PP descriptor with a formula comprising:
$$SD {1}^{2} = \frac{1}{2} SD Δ {G}^{2} ,$$
and
calculating a second PP descriptor with a formula comprising:
SD2²=2SD²−SD1², where, SDΔG is a standard deviation of the successive glucose values from respective CGM traces at (G_{t+Δt}−Gₜ) time intervals, and SD is the standard deviation of the glucose values.

10. The computer-implemented method of claim 9, further comprising calculating an area of a fitting ellipse characterized by SD1 and SD2, which represents a total variability for the individual according to a formula comprising:
$$S ( I ) = π SD 1 ( I ) SD 2 ( I ) .$$

11. A processor-implemented method, the method comprising:
identifying a concentration level of a metabolite corresponding to a subject;
transforming the concentration level into a symbol from a set of symbols, wherein the symbol represents a category of concentration levels;
identifying a matrix of transition probabilities between symbols in a series of symbols;
identifying an entropy rate of concentration level state categories based on the matrix; and
determining a diagnostic state for the subject based on the entropy rate.

12. The method of claim 11, wherein the metabolite is glucose, and wherein set of symbols contains 8 symbols.

13. The method of claim 11, wherein determining the diagnostic state comprises determining whether or not the subject is diabetic.

14. The method of claim 11, further comprising:
outputting a visualization of the matrix;
providing the visualization of the matrix to a user.

15. The method of claim 6, further comprising:
outputting a second visualization of a Poincare plot that corresponds to the visualization of the matrix.

16. A computer system, the computer system comprising:
one or more processors, one or more computer-readable memories, one or more computer-readable tangible storage medium, and program instructions stored on at least one of the one or more memories, wherein the computer system is capable of performing a method comprising:
identifying a concentration level of a metabolite corresponding to a subject;
transforming the concentration level into a symbol from a set of symbols, wherein the symbol represents a category of concentration levels;
identifying a matrix of transition probabilities between symbols in a series of symbols;
identifying an entropy rate of concentration level state categories based on the matrix; and
determining a diagnostic state for the subject based on the entropy rate.

17. The computer system of claim 16, wherein the metabolite is glucose, and wherein the set of symbols contains 8 symbols.

18. The computer system of claim 17, wherein determining the diagnostic state comprises determining whether or not the subject is diabetic.

19. The computer system of claim 18, wherein determining the diagnostic state comprises determining an effectiveness of a treatment.

20. The computer system of claim 16, wherein identifying a concentration level of a metabolite comprises continuous glucose monitoring of an individual.
